SEQUENCE LISTING (2 sequences)

SEQ ID NO: 1 - protein, 42 aa, human [sequence omitted]

SEQ ID NO: 2 - protein, 140 aa, human
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2010/0167320 A1
(43) Pub. Date: Jul. 1, 2010

(54) Compositions and Methods for Detecting and Quantifying Toxic Substances in Disease States

(75) Inventors: Hans T. Beernink, Camarillo, CA (US); Mary M. Brodey, Ventura, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/536,830

(22) Filed: Aug. 6, 2009

Related U.S. Application Data
(63) Continuation of application No. 11/669,105, filed on Jan. 30, 2007, now abandoned.
(60) Provisional application No. 60/763,247, filed on Jan. 30, 2006.

(51) Int. Cl. G01N 33/53; C12Q 1/26; C07K 2/00

(52) U.S. Cl. 435/7.92; 436/501; 435/25; 530/300

(57) ABSTRACT

The present invention relates to compositions comprising synthetic aggregated peptides (SAPs). The present invention also relates to the use of these SAPs as standards in methods for quantifying substances in a sample. The present invention also relates to methods of detecting, diagnosing and monitoring the progression of an abnormal condition in a subject with the methods comprising determining levels of an aggregated biomarker in a subject by measuring levels of the aggregated biomarker in the subject and correlating these levels to a standard curve, where the standard curve is established using a SAP peptide as the standard.

### CROSS REFERENCE TO RELATED APPLICATIONS

This application claims priority to U.S. Provisional Application No. 60/763,247, filed Jan. 30, 2006, the contents of which are incorporated by reference as if set forth fully herein.

### BACKGROUND OF THE INVENTION

1. Field of the Invention

The present invention relates to compositions comprising synthetic aggregate peptides (“SAP peptides or SAPs”). The present invention also relates to the use of these SAPs as standards in methods for quantifying substances in a sample. The present invention also relates to methods of detecting, diagnosing and monitoring the progression of an abnormal condition in a subject with the methods comprising determining levels of an aggregated biomarker in a subject by measuring levels of the aggregated biomarker in the subject and correlating these levels to a standard curve, where the standard curve is established using a SAP peptide as the standard. The present invention also provides a method of screening antibodies and chemical compounds as potential therapeutics developed for the treatment, prevention or diagnosis of abnormal conditions involving aggregated biomarkers.

2. Background of the Invention

Aggregated peptides are gaining recognition as potential toxins involved in a variety of disease states. For example, there is increasing evidence that soluble aggregates of beta-amyloid (Aβ) (1-42) may be responsible for neuronal cell death in Alzheimer's rather than plaques. Interestingly, Aβ is present in Alzheimer-related plaques formation, but it is present in the well-known fibrillized form as well as smaller oligomeric forms.

Similarly, aggregates of alpha-synuclein are being implicated in cell death associated with Parkinson's disease; aggregates of huntingtin peptide are being implicated in cell death associated with Huntington's Disease and aggregates of superoxide dismutase 1 are being implicated in cell death associated with amyotropic lateral sclerosis (ALS). Aggregates of prion protein are implicated in several prion diseases, such as bovine spongiform encephalopathy, variant Creutzfeldt-Jakob disease, Gerstmann-Strässler-Scheinke Syndrome, Fatal Familial Insomnia, kuru, scrapie, transmissible mink encephalopathy, chronic wasting disease of cervids, feline spongiform encephalopathy, exotic ungulate encephalopathy, and prion-mediated protein misfolding in yeast and other organisms. Aggregates of stefin B are implicated in myoclonus epilepsy. Aggregates of tau are implicated in frontotemporal dementia/tauopathy. Aggregates of transthyretin are implicated in senile systemic amyloidosis and familial amyloid polyneuropathy. Aggregates of ataxin-1 are implicated in spinocerebellar ataxia type-1. Aggregates of gelsolin are implicated in familial amyloidosis of the Finnish type. Aggregates of BRI are implicated in familial Brisith dementia. In fact, aggregated peptides are now being implicated in other disease states. In heart disease, aggregated HSP is implicated in desmin-related cardiomyopathy. Aggregates of alphaB crystallin are implicated in desmin-related cardiomyopathy, dilated cardiomyopathy, and hypertrophic cardiomyopathy. Aggregates of amylin as well as islet amyloid peptide are implicated in type II diabetes melletis. Aggregates of beta2-microglobulin are implicated in a systemic amyloidosis known as dialysis-related amyloidosis. Aggregates immunoglobulin light chain are implicated in a systemic amyloidosis known as light-chain amyloidosis. Aggregates of antithrombin are implicated in thrombosis. Protein aggregates are also implicated in cystic fibrosis, rheumatoid arthritis, and cirrhosis of the liver.

With the increased awareness that these aggregated proteins may be playing a role in disease states, it becomes increasingly important to accurately measure these aggregated peptides. Indeed, to aid in the diagnosis and monitoring of patients suffering from or at risk of suffering from a disease state characterized by the presence of these aggregated peptides, it is becoming critical to quantitatively assess levels of these aggregated peptides. To that end, peptide standards are needed to standardize and calibrate assays used to quantify these aggregated peptides.

There are, however, difficulties in obtaining pure forms of the aggregated peptides to use as standards in these developing assays. First, the general structure of the aggregated peptides themselves makes these compositions highly soluble. The high solubility of these aggregated peptides, in turn, makes it quite challenging to isolate large enough quantities of sufficiently purified aggregated peptides that can be used as standards in quantitative assays. Aggregated peptides are generally held together by non-covalent interaction, thus making the aggregates dynamic in size and complexity. Recent evidence, however, suggests that cytotoxic forms of aggregates may be more homogeneous in nature, yet purification and storage of these components is, in fact, complicated by the dynamic nature of aggregate assembly. In addition, these aggregated peptides often do not survive the freeze-thaw cycle, thus putting a damper on the number of assays that can be standardized with a single lot of isolated aggregated peptide.

What is needed in the art, therefore, is a synthetic standard that circumvents these difficulties in using isolated, naturally occurring aggregated peptides. The standards should be easy to synthesize, stable over time, stable within solution, and able to withstand repeated freeze-thaw cycles. In addition, it is critical that the synthetic standard present an epitope to an antibody or aptamer that is identical to or closely mimics the naturally occurring epitope present on the aggregated peptide.

### SUMMARY OF THE INVENTION

The present invention relates to methods for quantifying a known biomarker in a sample, with the methods comprising an assay that compares the binding activity of a binding agent towards the known biomarker with the binding activity of the binding agent towards a composition comprising a branched synthetic aggregate peptide (SAP peptide or SAP).

The present invention also relates to methods of detecting and diagnosing an abnormal condition is a subject, with the methods comprising detecting the binding activity of a binding agent towards at least one standard to establish a standard curve, where the standard comprises a SAP peptide. The methods further comprise contacting a sample from the subject with at least one binding agent that is capable of binding an aggregated biomarker, detecting the level binding activity of the binding agent in the sample and correlating the binding activity in the sample to the established standard curve to determine the levels of the aggregated biomarker in the subject. The determined levels of the aggregated biomarker, using SAP as a standard, are then compared to normal levels of the aggregated biomarker to determine if a difference exists between the measured levels of the aggregated biomarker and normal levels of the aggregated biomarker.

The present invention also relates to methods of monitoring the progression of an abnormal condition in a subject and methods of monitoring the efficacy of a treatment in a subject with an abnormal condition, with the methods comprising detecting the binding activity of a binding agent towards at least one standard to establish one or more standard curves, where the standard comprises a SAP peptide. The methods further comprise contacting more than one sample from a subject with at least one binding agent that is capable of binding an aggregated biomarker, where the multiple samples are taken from the subject at different time points. The level binding activity of the binding agent in the samples is detected and the binding activity in each sample is correlated to the established standard curve(s) to determine the levels of the aggregated biomarker in the subject. The determined levels of the aggregated biomarker from each time point, using SAP as a standard, are then compared to each other to determine if the measured levels of the aggregated biomarker are changing over time.

The present invention also relates to compositions comprising a branched SAP peptide, where at least one branch of the SAP peptide comprises the N-terminus of amyloid beta peptide.

### BRIEF DESCRIPTION OF THE DRAWINGS

FIG. 1 depicts one specific embodiment of the present invention, which is a 4-branched SAP peptide. The particular embodiment depicted is commonly referred to as a multiple antigenic peptide (or MAP). The MAP core comprises a β-alanine amino acid with a single lysine. The amino group of the β-alanine is attached through the α-carboxyl group of the lysine residue. The lysine provides two amino groups for attachment of additional residues. To each of the amine groups is attached an additional lysine residue, expanding the number of amine terminals to four. One or more peptide chains of interest can then be covalently attached to each of the 4 amino terminals. In this way, the MAP may comprise 4 separate peptide chains of interest. A MAP with 8 branches would be established by adding one additional layer of lysine residues to the 4 amino terminals prior to attachment of any peptide chains of interest. MAPs with 16, 32, 64 or more branches would be established by adding subsequent layers of lysine residues to the core structure prior to addition of any peptide(s) of interest.

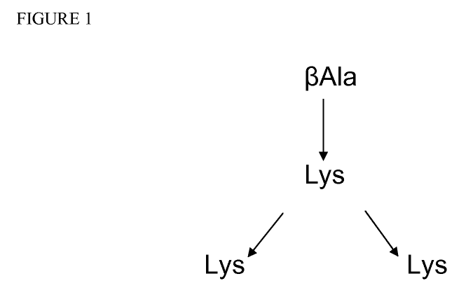

FIG. 2 depicts a standard curve generated using one embodiment of MAP peptides presented herein. The standard curve generated using the MAP peptide comprising the N-terminus (1-20) of the amyloid beta peptide on each of the 4 arms of the MAP (MAP-Aβ₁₋₂₀) closely mimics that standard curve generated using pure aggregated amyloid beta (1-42) peptide.

FIG. 3 depicts the results of an assay measuring levels of aggregated amyloid beta in subjects with a standard curve generated with MAP-Aβ₁₋₂₀ peptide.

FIG. 4 depicts the results of an assay measuring levels of aggregated amyloid beta in subjects with a standard curve generated with MAP-Aβ₁₋₂₀ peptide.

FIG. 5 depicts the time course for aggregation of alpha-synuclein in laboratory conditions at 37° C.

FIG. 6 depicts a standard curve generated using one embodiment of MAP peptides presented herein. The standard curve generated using the MAP peptide comprising a portion of the C-terminus of the alpha-synuclein peptide on each of the 4 arms of the MAP (MAP-alpha-synuclein) closely mimics that standard curve generated using laboratory-aggregated alpha-synuclein.

### DETAILED DESCRIPTION OF THE INVENTION

The present invention relates to methods for quantifying a known biomarker in a sample, with the methods comprising an assay that compares the binding activity of a binding agent towards the known biomarker with the binding activity of the binding agent towards a composition comprising a branched synthetic aggregate peptide (SAP).

As used herein, a sample can be any environment that may be suspected of containing the antigen of interest. Thus, a sample includes, but is not limited to, a solution, a cell or a portion thereof, tissue culture medium, a body fluid, a tissue or portion thereof, and an organ or portion thereof. Examples of cells include, but are not limited to, bacteria, yeast, plant, insect, avian, fish, reptilian, amphibian, and mammalian such as, for example, bovine, ovine, equine, porcine, canine, feline, human and nonhuman primates. Other examples include non-animal organisms that may harbor similar antigens of interest, include but are not limited to molds, viruses, and other model systems for the study of biological processes. The scope of the invention should not be limited by the cell type assayed or the media in which these cells are cultured or processed (e.g., for the production of cellular or tissue lysates). Examples of biological samples to be assayed include, but are not limited to, blood, plasma, serum, urine, saliva, milk, seminal plasma, synovial fluid, interstitial fluid, cerebrospinal fluid, lymphatic fluids, bile, and amniotic fluid, tissue culture medium, tissue homogenates, cell lysates, chemical solutions. The scope of the methods of the present invention should not be limited by the type of sample assayed. The terms “subject” “patient” and “organism” are used interchangeably herein and are used to mean any animal. In one embodiment the animal is a mammal. In a more particular embodiment, the animal is a human or nonhuman primate.

The samples may or may not have been removed from their native environment. Thus, the portion of sample assayed need not be separated or removed from the rest of the sample or from a subject that may contain the sample. For example, the blood of a subject may be assayed for the known biomarker without removing any of the blood from the patient. Of course, the sample may also be removed from its native environment. Furthermore, the sample may be processed prior to being assayed. For example, the sample may be diluted or concentrated; the sample may be purified and/or at least one compound, such as an internal standard, may be added to the sample. The sample may also be physically altered (e.g., centrifugation, size exclusion chromatography, size permeation chromatography, filtered, including ultrafiltration, affinity separation) or chemically altered (e.g., adding an acid, base, buffer, solvent, treating with a chemically reactive resin, heating) prior to or in conjunction with the methods of the current invention. Processing also includes freezing and/or preserving the sample prior to assaying, extracting secreted cellular products from surrounding medium, or physical disruption of cells and/or tissue to actively extract the analyte of interest.

As used herein the term SAP, or synthetic aggregated peptide, is used to mean a synthetic compound comprising a core component with one more peptide or single amino acid branches extending from the core. Unlike typical hapten-carrier complexes, e.g., keyhole limpet hemocyanin (KLH) where the carrier is generally immunogenic even in the absence of hapten, it is possible that the core of the SAPs of the present invention may be administered to an animal without causing a detectable immunogenic reaction. To be clear, the SAPs of the present invention will be useful in the methods of the present invention even if the core of the SAPs is able to cause a detectable immunogenic reaction. The peptides or amino acid arms extending from the core are referred to as the peptide(s) of interest. Examples of core components include, but are not limited to, amino acids, saccharides, oligosaccharides, polysaccharides, other polymers, such as but not limited to, polyethylene glycol, and any combination of the above. The invention should not be limited by the composition of the core, provided that the core is capable of attachment with one or more arms for the peptide(s) of interest. The attachment of the peptide arm to the SAP core may be covalent or non-covalent. In one embodiment, the core of the SAPs may comprise 2 or more branches or arms with the peptide(s) of interest attached thereto. In one embodiment, the SAP comprises 4 arms. In another embodiment, the SAP comprises more than 4 arms, such as, but not limited to, 8, 16, 32, 64 or more arms. It is conceivable that the SAPs may also comprise a number that is not an even power of 2, such as, but not limited to, 3, 5, 6, 7, 9, 10, 11, 12, 13, 14, 15, 17, 18, 19, 20 etc.

In one embodiment, the SAP comprises a core, where the core comprises one or more amino acids. In this particular embodiment, where the core comprises one or more amino acids, the SAP is commonly referred to as a multiple antigenic peptide, or MAP. Methods of producing MAPs are well known in the art. See Tam, J P, Proc. Nat'l Acad. Sci. USA, 85:5409-5413 (1988), which is incorporated by reference. In one specific embodiment, the MAP is 4-armed MAP with the core of the MAP comprising an amino acid, such as, but not limited to, (3-alanine that is attached to a first lysine reside via a typical peptide bond. This first lysine residue, i.e., the first layer of the MAP, provides 2 amino groups to which are attached a second and third lysine residue. These second and third lysine groups (the second layer of the MAP), in turn, provides 4 amino groups to which can be attached the peptide sequence(s) of interest.

Variations of this core structure are readily obtained by altering the number and identity of the amino acids that form the core of the MAP. For example, if a MAP with 3 arms is desired, the second layer of the MAP may, for example, comprise a second lysine residue and another residue that does not provide 2 amino groups. Examples of such amino acid residues include, but are not limited to alanine, valine, phenylalanine, methionine, leucine, isoleucine, aspartate, glutamate, serine, threonine, tyrosine, cysteine and other non-naturally occurring amino acids. In this way, the second layer terminates in 2 amino groups offered by the second lysine and a third amino group offered by the other amino acid residue.

To the core of the SAP is attached to the peptide arm or peptide arms. As used herein, a peptide arm means a peptide or amino acid that is intended to be incorporated onto the core of the SAP as an arm extending therefrom. A peptide, in turn, is used to mean a chain of 2 or more amino acids joined together by peptide bonds. Thus, for the purposes of the present invention, a peptide includes di-peptides, tri-peptides, oligopeptides, polypeptides, full length protein chains, and proteins. The length of the peptide arm may vary depending on the intended use and can be any size, provided that the binding agent can specifically bind the SAP.

In one embodiment, each arm of the SAP comprises an identical peptide arm. In another embodiment, each arm of the SAP comprises peptides of interest where the peptides are not identical to each other. For example, a SAP comprising 4 arms may possess 4, 3 or 2 non-identical peptides of interest. As used herein, the phrase “identical peptides of interest” means peptides chains that have the identical primary structure as well as any post-translational modifications, such as, but not limited to, glycosylation, oxidation, acetylation, methylation, phosphorylation, acylation, nitrosylation, citrullination. The “post-translational modifications” may be natural or they may be synthetic modifications that normally do not take place in a native cellular environment. For example, the peptides of interest or portions thereof may possess polyethylene glycol (PEG) (i.e., the peptide is PEGylated), be amidated with succinimyl ester or be cysteine alkylated. Additional protein modifications include, but are not limited to, ubiquinylation, prenylation and modifications resulting from the action of enzymes such as, but not limited to transglutaminase, and glutathione transferase. Thus, two peptide chains that are identical in amino acid sequence, but have, for example, different glycosylation patterns, different phosphorylation patterns are considered non-identical peptides of interest for the purposes of the present invention. For example, at least one arm of the SAP may comprise a peptide chain where the chain is unphosphorylated, and at least one arm of the SAP, where the peptide chain is phosphorylated. A single SAP may thus be used to monitor phosphorylation (or other enzymatic) events and/or may be used to determine proportions of phosphorylated (or differently modified) peptides within a system. Such other modifications include, but are not limited to cleavage events involving such enzymes as, but not limited to, proteases such as caspases and secretases. An example of a cleavage even includes, but is not limited to, the cleavage of Aβ1-42 to Aβ1-20.

The inventors have discovered that the SAPs can serve as standards in binding assays that employ binding agents that bind to known biomarkers. As used herein, the term binding agent is used to mean a composition that binds specifically to the known biomarker. Examples of binding agents include, but are not limited to, natural proteins such as receptors, antibodies and functional fragments thereof, as well as synthetic molecules, such as but not limited to, aptamers and protein fragments screened by phage-display or other methods. As used herein, the term “antibody” is used to mean immunoglobulin molecules and functional fragments thereof, regardless of the source or method of producing the fragment. As used herein, a “functional fragment” of an immunoglobulin is a portion of the immunoglobulin molecule that specifically binds to a binding target. Thus, the term “antibody” as used herein encompasses whole antibodies, such as antibodies with isotypes that include but are not limited to IgG, IgM, IgA, IgD, IgE and IgY, and even single-chain antibodies found in some animals e.g., camels. Whole antibodies may be monoclonal or polyclonal, and they may be humanized or chimeric. The term “monoclonal antibody” as used herein is not limited to antibodies produced through hybridoma technology. Rather the term “monoclonal antibody” refers to an antibody that is derived from a single clone, including any eukaryotic, prokaryotic, or phage clone, and not the method by which it is produced. The term “antibody” also encompasses functional fragments of immunoglobulins, including but not limited to Fab fragments, Fab′ fragments, F(ab′)₂ fragments and Fd fragments. “Antibody” also encompasses fragments of immunoglobulins that comprise at least a portion of a V_{L} and/or V_{H} domain, such as single chain antibodies, a single-chain Fv (scFv), disulfide-linked Fvs and the like.

The antibodies used in the present invention may be monospecific, bispecific, trispecific or of even greater multispecificity. In addition the antibodies may be monovalent, bivalent, trivalent or of even greater multivalency. Furthermore, the antibodies of the invention may be from any animal origin including, but not limited to, birds and mammals. In specific embodiments, the antibodies are human, murine, rat, sheep, rabbit, goat, guinea pig, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described in U.S. Pat. No. 5,939,598, which is herein incorporated by reference.

The antibodies used in the present invention may be described or specified in terms of the epitope(s) or portion(s) of a polypeptide to which they recognize or specifically bind. Or the antibodies may be described based upon their ability to bind to specific conformations of the antigen, or specific modification (e.g., cleavage or chemical, natural or otherwise, modification of sequence). In one embodiment, a single antibody used in the methods of the present invention is specific towards an epitope presented on a SAP and towards an epitope presented on the known biomarker that is being assayed.

The specificity of the antibodies used in present invention may also be described or specified in terms of their binding affinity towards the antigen (epitope) or of by their cross-reactivity. Specific examples of binding affinities encompassed in the present invention include but are not limited to those with a dissociation constant (Kd) less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In one embodiment, the antibody that is used in the methods of the present invention has a substantially equivalent binding affinity towards the epitope presented on a SAP and towards an epitope presented on the known biomarker that is being assayed. As used herein, a substantially equivalent binding affinity means within the same order of magnitude of the dissociation constant.

The antibodies used in the invention also include derivatives that are modified, for example, by covalent attachment of any type of molecule to the antibody such that covalent attachment does not prevent the antibody from generating an anti-idiotypic response. Examples of modifications to antibodies include but are not limited to, glycosylation, acetylation, pegylation, phosphorylation, amidation, derivatization by known protecting/blocking groups, proteolytic cleavage, linkage to a cellular ligand or other composition, such as a signaling moiety, a label etc. In addition, the antibodies may be linked or attached to solid substrates, such as, but not limited to, beads, particles, glass surfaces, plastic surfaces, ceramic surfaces, metal surfaces. Any of numerous chemical modifications may be carried out by known techniques, including, but not limited to, specific chemical cleavage, acetylation, biotinylation, farnesylation, formylation, inhibition of glycosylation by tunicamycin and the like. Additionally, the derivative may contain one or more non-classical or synthetic amino acids.

The antibodies used in the present invention may be generated by any suitable method known in the art. Polyclonal antibodies can be produced by various procedures well known in the art. For example, a SAP or an epitope on the SAP can be administered to various host animals including, but not limited to, rabbits, goats, chickens, mice, rats, to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (both of which are incorporated by reference).

Methods for producing and screening for specific antibodies using hybridoma technology are routine and well known in the art such as, but not limited to, immunizing a mouse, hamster, or rat. Additionally, newer methods to produce rabbit and other mammalian monoclonal antibodies may be available to produce and screen for antibodies. In short, methods of producing and screening antibodies, and the animals used therein, should not limit the scope of the invention. Once an immune response is detected, the mouse spleen is harvested and splenocytes isolated. The splenocytes are then fused by well known techniques to any suitable myeloma cells, for example cells from cell line SP2/0 available from the ATCC. Hybridomas are selected and cloned by limited dilution. The hybridoma clones can then be assayed by methods known in the art for cells that secrete antibodies capable of binding a biomarker of the present invention. Ascites fluid, which generally contains high levels of antibodies, can be generated by immunizing mice with positive hybridoma clones. In addition, antibodies can be produced using a variety of alternate methods, including but not limited to bioreactors and standard tissue culture methods, to name a few.

The antibodies used the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library. Phage expressing an antigen binding domain that binds the antigen of interest can be selected or identified with the antigen of interest, such as using a labeled antigen or antigen bound or captured to a solid surface or bead. The phage used in these methods are typically filamentous phage including, but not limited to, fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108, all of which are incorporated by reference.

Antibody fragments which recognize specific epitopes may be generated by known techniques. For example, Fab and F(ab)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

Other methods, such as recombinant techniques, may be used to produce Fab, Fab′ and F(ab′)₂ fragments and are disclosed in PCT publication WO 92/22324; Mullinax et al., BioTechniques 12(6):864-869 (1992); and Sawai et al., AJRI 34:26-34 (1995); and Better et al., Science 240:1041-1043 (1988), which are herein incorporated by reference. After phage selection, for example, the antibody coding regions from the phage can be isolated and used to generate whole antibodies, including human antibodies, or any other desired antigen binding fragment, and expressed in any desired host, including mammalian cells, insect cells, plant cells, yeast, and bacteria.

Examples of techniques which can be used to produce other types of fragments, such as scFvs and include those described in U.S. Pat. Nos. 4,946,778 and 5,258,498; Huston et al., Methods in Enzymology 203:46-88 (1991); Shu et al., Proc. Nat'l Acad. Sci. (USA) 90:7995-7999 (1993); and Skerra et al., Science 240:1038-1040 (1988), all of which are incorporated by reference. For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, all of which are herein incorporated by reference. Humanized antibodies are antibody molecules from non-human species antibody that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See U.S. Pat. No. 5,585,089; Riechmann et al., Nature 332:323 (1988), both of which are herein incorporated by reference. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Nat'l. Acad. Sci. 91:969-913 (1994)), and chain shuffling (U.S. Pat. No. 5,565,332), all of which are hereby incorporated by reference.

Completely human antibodies may be particularly desirable for therapeutic treatment or diagnosis of human patients. Human antibodies can be made by a variety of methods known in the art including phage display methods described above using antibody libraries derived from human immunoglobulin sequences. See also. U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated by reference.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995), which is hereby incorporated by reference. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference.

Still another approach for generating human antibodies utilizes a technique referred to as guided selection. In guided selection, a selected non-human monoclonal antibody, e.g., a mouse antibody, is used to guide the selection of a completely human antibody recognizing the same epitope. (Jespers et al., Biotechnology 12:899-903 (1988), herein incorporated by reference).

Accordingly, using the binding agents and SAP standards described herein, the present invention provides methods of detecting and quantifying a known biomarker in a sample, with the methods comprising contacting the sample with a binding agent that is specific for both the known biomarker and the SAP standard, and detecting the binding of the binding agent to the known biomarker and SAP standard. The binding agent may be coated onto a cell culture surface or a 96-well plate, such as an ELISA plate, or the capture antibody may be bound to or coated on beads or columns, or any surface or environment capable of housing the capture antibody such that it is available to bind to the antigen of interest.

Examples of an assay used in the methods of the present invention to assess the quantity of a known biomarker include, but are not limited to, immunosorbence assays and competitive binding assays. Specific embodiments of some of the assays listed include, but are not limited to, direct and indirect assays, as well as binary and tertiary sandwich assays. In one embodiment, the assay is an immunosorbence assay. In more specific embodiments, the immunosorbence assay is a colorimetric assay, an enzyme-linked immunosorbence assay (ELISA), a planar array or a radioimmunoassay. Other examples of assays that may be used in the methods of the present invention include, but are not limited to, bead or particle-based immunoassays, chemiluminescient assays, surface plasmon resonance (SPR) based assays, fluorescence assays, rolling-circle amplification assays, assays using dendrimers, and other enzyme or non-enzymatic amplification schemes.

The methods of the present invention utilize SAPs as standards to quantify and standardize assays that are designed to quantify known biomarkers. The invention is not limited to the identity or class of known biomarkers. Examples of classes of biomarkers include but are not limited to, carbohydrates such as monosaccharides, disaccharides, oligosaccharides and polysaccharides, proteins, peptides and amino acids, including, but not limited to, oligopeptides, polypeptides and mature proteins, nucleic acids, oligonucleotides, polynucleotides, lipids, fatty acids, lipoproteins, proteoglycans, carbohydrates, glycoproteins, organic compounds, inorganic compounds, ions, and synthetic and natural polymers, peptides, proteins, sacchraides, carbohydrates.

In one embodiment, the biomarker is a peptide. Examples of biomarker peptides include but are not limited to, beta amyloid (Aβ), huntingtin peptide, alpha-synuclein, tau, superoxide-dismutase 1 (SOD-1), prion peptide, stefin B, transthyretin, ataxin-1, gelsolin, BR1, HSP, alphaB crystalline, amylin, beta2-microglobulin, immunoglobulin light chain, antithrombin, and portions thereof. The phrase “portion of a peptide” is readily understood in the art. The above listed peptides are well-known for their association with disease states. For example, Aβ is associated with Alzheimer's Disease, alpha-synuclein is associated with Parkinson's Disease and Alzheimer's Disease, SOD-1 is associated with amytropic lateral sclerosis (ALS), huntingtin is associated with Huntington's Disease, and prion is associated with Creutzfeldt-Jakob Disease and other spongiform encephalopathies.

In a more specific embodiment, the biomarker is an aggregated peptide. As used herein, an aggregated peptide is an aggregation of peptides that form a distinct globular, ball-like structure, or annular structure. The aggregated peptide is thought to form by an initial nucleation process where hydrophobic regions of the individual peptide chains aggregate in the center of the globule to form a hydrophobic core. The aggregated core then polymerizes additional peptide chains onto the core. In general, the aggregated peptide will polymerize until it forms a stable globular or annular structure with a hydrophobic core and hydrophilic surface. Once the stable aggregated peptide forms, the aggregated peptide will, in general, cease polymerization. The structure of the aggregated peptide accounts for its generally high solubility and stability. An example of an aggregated peptide is illustrated in Barghorn, S. et al., J. Neurochem. 95(3):834-47 (2005), which is incorporated by reference. For example, aggregated Aβ peptide (Aβ₁₋₄₂) is gaining attention as a potential toxin that is associated with Alzheimer's Disease. Similarly, aggregated forms of huntingtin peptide, alpha-synuclein, superoxide-dismutase 1 (SOD-1) and prion peptide are gaining attention as potential toxins in Huntington's Disease, Parkinson's Disease, ALS, and Creutzfeld-Jakob Disease, respectively. In particular the compositions and methods of the present invention can be used in any abnormal condition that may be characterized by amyloidogenesis. Table 1 and 2 list a representative of diseases attributed to toxic protein aggregates, the list is not intended to be inclusive as many other disease are also know to be attributed to toxic protein aggragates.

TABLE 1

| Disease | Protein | Reference |
|---|---|---|
| Alzheimer's disease | beta amyloid | Lambert, M., et al. (1998) PNAS |
| | | 95: 6448. |
| | | Kayed, R., et al. (2003) Science |
| | | 300: 486. |
| | | Demuro, A., et al. (2005) J. Biol. |
| | | Chem. 280: 17294. |
| Parkinson's disease | alpha-synuclein | El-Agnaf, A., et al. (2006) |
| | | FASEB J. 20: 419. |
| Huntington's disease | huntingtin peptide | Demuro, A., et al. (2005) J. Biol. |
| | | Chem. 280: 17294. |
| Amyotropic lateral sclerosis (ALS) | superoxide | Cleveland, D. W. and R. J. Rothstein |
| | dismutase 1 | (2001) Nat. Rev. |
| | | Neurosci. 2: 806. |
| Bovine spongiform encephalopathy, | prion | Demuro, A., et al. (2005) J. Biol. |
| variant Creutzfeldt-Jakob disease | | Chem. 280: 17294. |
| Myoclonus epilepsy | stefin B | Lalioti, M. D., et al. (1997) |
| | | Nature 286: 767. |
| Frontotemporal dementia/tauopathy | tau | Spillantini, M. G. and M. Goedert |
| | | (1998) Trends Neurosci. 21: 428. |
| Senile systemic amyloidosis and | transthyretin | Quintas, A., et al. (1997) FEBS |
| familial amyloid polyneuropathy | | Lett. 418: 297-300. |
| Spinocerebellar ataxia type-1 | ataxin-1 | de Chiara, C., et al. (2005) J. |
| | | Mol. Biol. 354: 883. |
| Familial amyloidosis of the | gelsolin | Huff, M. E., et al. (2003) J. Mol. |
| Finnish type | | Biol. 334: 119. |
| Familial British dementia | BRI | El-Agnaf, O. M., et al. (2001) |
| | | Biochemistry 40: 3449. |

TABLE 2
Protein Aggregates in Other Diseases

| Disease | Protein | Reference |
|---|---|---|
| Familial Mediterranean | serum amyloid A | Van der Hilst, J. C., et al. (2005) |
| fever, systemic AA | | Clin. Exp. Med. 5: 87. |
| amyloidosis, visceral | | |
| amyloidosis | | |
| Desmin-related | alphaB crystallin/HSP | Atsushi Sanbe, A., et al. (2005) |
| cardiomyopathy, dilated | | PNAS 102: 13592. |
| cardiomyopathy, and | | Kumarapeli, A. R. and X. Wang |
| hypertrophic | | (2004). J. Mol. Cell |
| cardiomyopathy | | Cardiol. 376: 1097. |
| Diabetes | islet amyloid polypeptide | Demuro, A., et al. (2005) J. Biol. |
| | | Chem. 280: 17294. |
| Dialysis-related | beta2-microglobulin | Buxbaum, J. N. (2004) Curr. |
| amyloidosis | | Opin. Rheumatol. 16: 67. |
| Light-chain amyloidosis | immunoglobulin light chain | Buxbaum, J. N. (2004) Curr. |
| | | Opin. Rheumatol. 16: 67. |
| Senile systemic | transthyretin | Buxbaum, J. N. (2004) Curr. |
| amyloidosis | | Opin. Rheumatol. 16: 67. |
| Thrombosis | antithrombin | Corral, J., et al. (2005) |
| | | Haematologica 90: 238. |
| Cirrhosis of the liver | antitrypsin | Corral, J., et al. (2005) |
| | | Haematologica 90: 238. |
| Emphysema | serpine family of proteinase | Lomas, D. A. and R. W Carrell |
| | inhibitors | (2002) Nat. Rev. Genet. 3: 759. |
| Hereditary Systemic | Lysozyme | Pepys, M. B., et al. (1993) Nature |
| Amyloidosis | | 362: 553. |

In one embodiment, the methods of the present invention are directed towards the quantification of a known aggregated peptide as the biomarker. Thus, the binding agent must be capable of specifically binding the known aggregated biomarker. To quantify the binding activity of the binding agent towards the known aggregated biomarker, the methods depend upon the use of a SAP as a standard. As discussed, the arms of the SAP comprise a peptide arm. In a specific embodiment, the SAP comprises at least a portion of the same peptide that makes up the aggregated peptide as the biomarker. Thus, if the biomarker to be quantified is aggregated Aβ, the SAP may comprise at least a portion of the Aβ peptide on at least one arm of the SAP. In one specific embodiment, aggregated Aβ₁₋₄₂ is the known biomarker and the SAP standard comprises a hydrophilic portion of the Aβ peptide on each of 4 arms of the SAP. In a more specific embodiment, the SAP comprises the N-terminus of Aβ₁₋₄₂. In an even more specific embodiment, the SAP comprises at least 6 contiguous amino acids from amino acids 1-20 of SEQ ID NO. 1. In other specific embodiments, the SAP comprises at least 7, 8, 9, 10, 11, 12, 13, 14, 15, 16, 17, 18, 19 and 20 contiguous amino acids from amino acids 1-20 of SEQ ID NO. 1, below. The amino acid sequence of SEQ ID NO.1 represents the amino acid sequence of human Aβ₁₋₄₂ peptide.

SEQ ID NO.1: daefrhdsgy evhhgklvff aedvgsnkga iiglmvggvv ia

In another specific embodiment aggregated huntingtin peptide is the biomarker and the SAP standard comprises a portion of the huntingtin peptide on at least one arm of the SAP standard. The full length huntingtin peptide can be accessed under GenBank Accession No NM 002111, which is hereby incorporated by reference, and the SAP standard may comprise any portion of the huntingtin peptide. Furthermore, “huntingtin peptide”, as used herein indicates a peptide with an amino acid sequence that is at least 80%, 85%, 90%, 95%, 96%, 97%, 98% or 99% identical to the huntingtin peptide as disclosed by GenBank Accession No. NM 002111. The “huntingtin peptide” may also include the expanded poly-glutamine tracts that characterize the toxic protein species found in Huntington's disease. In another specific embodiment, the SAP standard comprises a portion of the huntingtin peptide on each arm of the SAP. In a more specific embodiment, the SAP standard comprises a portion of the N-terminus of the huntingtin peptide on one or more arms of the SAP and may include expanded polyglutamine tracts or portions thereof. In other specific embodiments, the SAP standard comprises a portion of the center or the C-terminus of the huntingtin peptide on one or more arms of the SAP.

In another specific embodiment aggregated alpha-synuclein peptide is the biomarker and the SAP standard comprises a portion of the alpha-synuclein peptide on at least one arm of the SAP standard. The full length human alpha-synuclein peptide and splice variants can be accessed under GenBank Accession Nos. P37840, NM 000345, NM 0077308, NP 009292, NP 000336, which are hereby incorporated by reference, and the SAP standard may comprise any portion of the alpha-synuclein peptide. Furthermore, “alpha-synuclein peptide,” as used herein indicates a peptide with an amino acid sequence that is at least 80%, 85%, 90%, 95%, 96%, 97%, 98% or 99% identical to the alpha-synuclein peptide as disclosed by GenBank Accession Nos. P37840, NM 000345, NM 0077308, NP 009292, NP 000336. In another specific embodiment, the SAP standard comprises a portion of the alpha-synuclein peptide on each arm of the SAP. In a more specific embodiment, the SAP standard comprises a portion of the C-terminus of the alpha-synuclein peptide on one or more arms of the SAP. In an even more specific embodiment, the SAP standard comprises at least 7, 8, 9, 10, 11, 12, 13, 14, 15, 16, 17, 18, 19 and 20 contiguous amino acids of SEQ ID NO:2, below. In particular, the SAP standard comprises amino acids 116-130 of SEQ ID NO:2, where SEQ ID NO:2 represents the amino acid sequence of human alpha-synuclein. In other specific embodiments, the SAP standard comprises a portion of the center or the N-terminus of the alpha-synuclein peptide on one or more arms of the SAP.

(SEQ ID NO:2)

```
    1 mdvfmkglsk akegvvaaae ktkqgvaeaa gktkegvlyv gsktkegvvh gvatvaektk
  61 eqvtnvggav vtgvtavaqk tvegagsiaa atgfvkkdql gkneegapqe giledmpvdp
121 dneayempse egyqdyepea
```

In another specific embodiment aggregated SOD-1 peptide is the biomarker and the SAP standard comprises a portion of the SOD-1 peptide on at least one arm of the SAP standard. The full length human SOD-1 peptide can be accessed under GenBank Accession Nos. NM 000454 and NC 000021, which are hereby incorporated by reference, and the SAP standard may comprise any portion of the SOD-1. Furthermore, “SOD-1 peptide,” as used herein indicates a peptide with an amino acid sequence that is at least 80%, 85%, 90%, 95%, 96%, 97%, 98% or 99% identical to the SOD-1 peptide as disclosed by GenBank Accession Nos. NM 000454 and NC 000021. In another specific embodiment, the SAP standard comprises a portion of the SOD-1 peptide on each arm of the SAP. In a more specific embodiment, the SAP standard comprises a portion of the N-terminus of the SOD-1 peptide on one or more arms of the SAP. In other specific embodiments, the SAP standard comprises a portion of the center or the C-terminus of the SOD-1 peptide on one or more arms of the SAP.

In another specific embodiment aggregated prion peptide is the biomarker and the SAP standard comprises a portion of the prion peptide on at least one arm of the SAP standard. The full length human prion peptide can be accessed under GenBank Accession No. P04156, which is hereby incorporated by reference, and the SAP standard may comprise any portion of the prion peptide. Furthermore, “prion peptide,” as used herein indicates a peptide with an amino acid sequence that is at least 80%, 85%, 90%, 95%, 96%, 97%, 98% or 99% identical to the prion peptide as disclosed by GenBank Accession No. P04156. In another specific embodiment, the SAP standard comprises a portion of the prion peptide on each arm of the SAP. In a more specific embodiment, the SAP standard comprises a portion of the N-terminus of the prion peptide on one or more arms of the SAP. In other specific embodiments, the SAP standard comprises a portion of the center or the C-terminus of the prion peptide on one or more arms of the SAP.

In another specific embodiment aggregated islet amyloid polypeptide is the biomarker and the SAP standard comprises a portion of the islet amyloid polypeptide on at least one arm of the SAP standard. The full length human islet amyloid polypeptide can be accessed under GenBank Accession No. NM 000415, which is hereby incorporated by reference, and the SAP standard may comprise any portion of the islet amyloid polypeptide. Furthermore, “islet amyloid polypeptide,” as used herein indicates a peptide with an amino acid sequence that is at least 80%, 85%, 90%, 95%, 96%, 97%, 98% or 99% identical to the islet amyloid polypeptide as disclosed by GenBank Accession No. NM 000415. In another specific embodiment, the SAP standard comprises a portion of the islet amyloid polypeptide on each arm of the SAP. In a more specific embodiment, the SAP standard comprises a portion of the N-terminus of the islet amyloid polypeptide on one or more arms of the SAP. In other specific embodiments, the SAP standard comprises a portion of the center or the C-terminus of the islet amyloid polypeptide on one or more arms of the SAP.

As used herein, “identity” is a measure of the identity of nucleotide sequences or amino acid sequences compared to a reference nucleotide or amino acid sequence, usually a wild-type sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, New Jersey (1994); von Heinje, G., Sequence Analysis In Molecular Biology, Academic Press (1987); and Sequence Analysis Primer, Gribskov, M. and Devereux, J., eds., M Stockton Press, New York (1991)). While several methods exist to measure identity between two polynucleotide or polypeptide sequences, the term “identity” is well known in the art (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam J Applied Math 48:1073 (1988). Computer programs may also contain methods and algorithms that calculate identity and similarity. Examples of computer program methods to determine identity and similarity between two sequences include, but are not limited to, GCS program package (Devereux, J., et al., Nucleic Acids Research 12(i):387 (1984)), BLASTP, BLASTN, FASTA (Atschul, S. F., et al., J Molec Biol 215:403 (1990)).

A polypeptide having an amino acid sequence at least, for example, about 95% “identical” to a reference nucleotide sequence encoding a peptide of interest, for example Aβ, is understood to mean that the amino acid sequence of the peptide is identical to the reference sequence except that the amino acid sequence may include up to about five mutations per each 100 amino acids of the reference peptide sequence encoding the Aβ peptide being used as the reference sequence. In other words, to obtain a polypeptide having an amino acid sequence at least about 95% identical to a reference amino acid sequence, up to about 5% of the amino acids in the reference sequence may be deleted or substituted with another amino acid, or a number of amino acids up to about 5% of the total amino acids in the reference sequence may be inserted into the reference sequence. These mutations of the reference sequence may occur at the N- or C-terminal positions of the reference amino acid sequence or anywhere between those terminal positions, interspersed either individually among amino acids in the reference sequence or in one or more contiguous groups within the reference sequence.

In another embodiment, the peptide arm(s) is (are) intended to mimic the structural motif, e.g., alpha helices, beta sheets, etc., of the aggregated biomarker, rather than the peptide sequence of the aggregated peptide. The secondary and tertiary structural motifs of proteins can be readily determined using current technology, and peptide arms can also be designed to mimic the target structural motif(s) of the aggregated peptides.

Of course, the methods of detecting known biomarkers can be combined with detecting other biomarkers that are also indicative of particular disease states or abnormal conditions. For example, the methods of the present invention can be combined with methods of detecting biomarkers such as, but not limited to, tau protein and cytokines, to name a few. In fact, the SAP compositions of the present invention may be used as standards in multiplex assays, where more than one biomarker is being assayed. In one embodiment, aggregates composed of more than one biomarker is being assayed, and a single SAP standard is used to calibrate or standardize the muliplex assay, where the single SAP comprises at least two non-identical peptides of interest.

The present invention also relates to methods of detecting and diagnosing an abnormal condition in a subject. As used herein, an abnormal condition indicates that the subject is exhibiting one or more signs not present in a normal, healthy individual. The signs of the abnormal condition may be asymptomatic, in that none of the signs are readily apparent to the subject or healthcare provider in the absence of testing. Of course, the abnormal condition may also manifest itself in one or more signs that are readily apparent to the subject or healthcare provider.

The methods of detecting and diagnosing an abnormal condition in a subject comprise detecting the binding activity of a binding agent towards at least one concentration of at least one standard to establish a standard curve, where the standard comprises a SAP peptide. Methods of generating a standard curve are well known in the art. In general, establishing a standard curve involves detecting the levels of binding activity of the binding agent to various known concentrations of the SAP standard. The curve is then generated by plotting the levels of binding activity against the known concentrations of SAP standards. The curve may be generated by simply plotting the coordinates on an appropriate graph, or the curve may be generated using an algorithm to compute the equation of the curve. The standard curve can be any shape, including but not limited to linear, parabolic, hyperbolic and sigmoidal.

The methods further comprise contacting a sample from the subject with at least one binding agent that is capable of binding an aggregated biomarker, detecting the level binding activity of the binding agent in the sample and correlating the binding activity in the sample to the established standard curve to determine the levels of the aggregated biomarker in the subject.

The invention is not limited by the method of detecting the binding of the binding agent to the biomarker and/or SAP standard. The detection method may require a specific label, or may be label-independent as in SPR, TRF, interferometry, nephelometry, or waveguide biorefringence interferometry. For example, the detection of binding may include, but is not limited to, using a second detection antibody that binds to the binding agent-biomarker complex, such as in a “sandwich ELISA,” using spectroscopy, such as mass spectroscopy or fluorescence spectrophotometry, and electrophoresis or other separation method, such as Western Blotting, chromatography, capillary electrophoresis, capillary immunodetection, or other separation-based methods. The use of subsequent detection antibodies to detect binding of the binding agent to the biomarker may include, but is not limited to, radioactive isotopes and enzymes, such as horse radish peroxidase or alkaline phosphatase, as has been described herein. Additionally, if the binding agent, for example, is bound to a bead or particle, methods of detecting and measuring bound antigen may also include flow cytometry (FACS), colorimetric or other “encoded” particle technologies, or magnetic separation technologies.

In ELISAs, the capture molecule, i.e., the binding agent that initially binds to the biomarker does not have to be conjugated to a label; instead, a labeled subsequent detection molecule (which may recognize the capture molecule) may be added to the well. One of skill in the art would be knowledgeable as to the parameters that can be modified to increase the signal detected as well as other variations of ELISAs known in the art. As used herein the term “capture molecule” is used mean a binding agent that immobilizes the biomarker by its binding to the biomarker. Further, a biomarker is “immobilized” if the biomarker or biomarker-capture molecule complex is separated or is capable of being separated from the remainder of the sample. When the capture molecule is coated to a well or other surface, a detection molecule may be added following the addition of the biomarker of interest to the wells. As used herein, a detection molecule is used to mean a molecule, such as an antibody or receptor, comprising a label. In a specific embodiment, the methods of the present invention comprise the use of a capturing antibody and a detection antibody to detect the biomarker. In a more specific embodiment, the capture antibody and the detection antibody are the same antibodies with the same binding specificities. In another specific embodiment, the capture antibody and the detection antibody are different antibodies.

A label, as used herein, is intended to mean a chemical compound or ion that possesses or comes to possess or is capable of generating a detectable signal. The labels of the present invention may be conjugated to the primary binding agent, e.g., primary antibody, or secondary binding agent, e.g., secondary antibody, the biomarker or a surface onto which the label and/or binding agent is attached. Examples of labels includes, but are not limited to, radiolabels, such as, for example, ³H and ³²P, that can be measured with radiation-counting devices; pigments, biotin, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems. Additional examples of labels include, but are not limited to, a phosphorescent dye, a tandem dye and a particle. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label also includes a “tag” or hapten that can bind selectively to a conjugated molecule such that the conjugated molecule, when added subsequently along with a substrate, is used to generate a detectable signal. For example, one can use biotin as a label and subsequently use an avidin or streptavidin conjugate of horseradish peroxidate (HRP) to bind to the biotin label, and then use a colorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their colorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9^{th} edition, CD-ROM, (September 2002), which is herein incorporated by reference.

A fluorophore of the present invention is any chemical moiety that exhibits an absorption maximum beyond 280 nm, and when covalently attached to a labeling reagent retains its spectral properties. Fluorophores of the present invention include, without limitation; a pyrene (including any of the corresponding derivative compounds disclosed in U.S. Pat. No. 5,132,432, incorporated by reference), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Ser. Nos. 09/968,401 and 09/969,853, incorporated by reference), a carbocyanine (including any corresponding compounds in U.S. Ser. Nos. 09/557,275; 09/969,853 and 09/968,401; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1, incorporated by reference), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896, incorporated by reference), a xanthene (including any corresponding compounds disclosed in U.S. Pat. Nos. 6,162,931; 6,130,101; 6,229,055; 6,339,392; 5,451,343 and U.S. Ser. No. 09/922,333, incorporated by reference), an oxazine (including any corresponding compounds disclosed in U.S. Pat. No. 4,714,763, incorporated by reference) or a benzoxazine, a carbazine (including any corresponding compounds disclosed in U.S. Pat. No. 4,810,636, incorporated by reference), a phenalenone, a coumarin (including an corresponding compounds disclosed in U.S. Pat. Nos. 5,696,157; 5,459,276; 5,501,980 and 5,830,912, incorporated by reference), a benzofuran (including an corresponding compounds disclosed in U.S. Pat. Nos. 4,603,209 and 4,849,362, incorporated by reference) and benzphenalenone (including any corresponding compounds disclosed in U.S. Pat. No. 4,812,409, incorporated by reference) and derivatives thereof. As used herein, oxazines include resorufins (including any corresponding compounds disclosed in 5,242,805, incorporated by reference), aminooxazinones, diaminooxazines, and their benzo-substituted analogs.

When the fluorophore is a xanthene, the fluorophore is optionally a fluorescein, a rhodol (including any corresponding compounds disclosed in U.S. Pat. Nos. 5,227,487 and 5,442,045, incorporated by reference), or a rhodamine (including any corresponding compounds in U.S. Pat. Nos. 5,798,276; 5,846,737; U.S. Ser. No. 09/129,015, incorporated by reference). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171, incorporated by reference). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Fluorophores for use in the invention include, but are not limited to, xanthene (rhodol, rhodamine, fluorescein and derivatives thereof) coumarin, cyanine, pyrene, oxazine and borapolyazaindacene. Most preferred are sulfonated xanthenes, fluorinated xanthenes, sulfonated coumarins, fluorinated coumarins and sulfonated cyanines. The choice of the fluorophore attached to the labeling reagent will determine the absorption and fluorescence emission properties of the labeling reagent and immuno-labeled complex. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

In one aspect of the invention, the fluorophore has an absorption maximum beyond 480 nm. In a particularly useful embodiment, the fluorophore absorbs at or near 488 nm to 514 nm (particularly suitable for excitation by the output of the argon-ion laser excitation source) or near 546 nm (particularly suitable for excitation by a mercury arc lamp).

Many of fluorophores can also function as chromophores and thus the described fluorophores are also preferred chromophores of the present invention.

In addition to fluorophores, enzymes also find use as labels. Enzymes are desirable labels because amplification of the detectable signal can be obtained resulting in increased assay sensitivity. The enzyme itself may not produce a detectable signal but is capable of generating a signal by, for example, converting a substrate to produce a detectable signal, such as a fluorescent, colorimetric or luminescent signal. Enzymes amplify the detectable signal because one enzyme on a labeling reagent can result in multiple substrates being converted to a detectable signal. This is advantageous where there is a low quantity of target present in the sample or a fluorophore does not exist that will give comparable or stronger signal than the enzyme. The enzyme substrate is selected to yield the preferred measurable product, e.g. colorimetric, fluorescent or chemiluminescence. Such substrates are extensively used in the art, many of which are described in the MOLECULAR PROBES HANDBOOK, supra.

In a specific embodiment, a colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including Amplex® Red reagent and its variants (U.S. Pat. No. 4,384,042) and reduced dihydroxanthenes, including dihydrofluoresceins (U.S. Pat. No. 6,162,931, incorporated by reference) and dihydrorhodamines including dihydrorhodamine 123. Peroxidase substrates that are tyramides (U.S. Pat. Nos. 5,196,306; 5,583,001 and 5,731,158, incorporated by reference) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

Another preferred colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912, incorporated by reference) fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986, incorporated by reference).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. Preferred fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236, incorporated by reference), 4-methylumbelliferyl beta-D-galactopyranoside, carboxyumbelliferyl beta-D-galactopyranoside and fluorinated coumarin beta-D-galactopyranosides (U.S. Pat. No. 5,830,912, incorporated by reference).

Additional enzymes include, but are not limited to, hydrolases such as cholinesterases and peptidases, oxidases such as glucose oxidase and cytochrome oxidases, and reductases for which suitable substrates are known.

Specific embodiments of the present invention comprise enzymes and their appropriate substrates to produce a chemiluminescent signal, such as, but not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

Additional embodiments comprise haptens such as biotin. Biotin is useful because it can function in an enzyme system or fluorogenic system to further amplify the detectable signal, and it can function as a tag to be used in affinity chromatography for isolation purposes. For detection purposes, an enzyme conjugate that has affinity for biotin is used, such as avidin-HRP or streptavidin-HRP. Subsequently a peroxidase substrate is added to produce a detectable signal. Alternatively, a colorimetric or fluorimetric reporter dye or protein that has affinity for biotin is used, such as streptavidin-R-Phycoerythrin.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

Fluorescent proteins also find use as labels for the labeling reagents of the present invention. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorophore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556, incorporated by reference, and the sulforhodamine fluorophores disclosed in U.S. Pat. No. 5,798,276, or the sulfonated cyanine fluorophores disclosed in U.S. Ser. Nos. 09/968/401 and 09/969/853, incorporated by reference; or the sulfonated xanthene derivatives disclosed in U.S. Pat. No. 6,130,101, incorporated by reference and those combinations disclosed in U.S. Pat. No. 4,542,104, incorporated by reference. Alternatively, the fluorophore functions as the energy donor and the fluorescent protein is the energy acceptor.

In one embodiment, the label is a fluorophore selected from the group consisting of fluorescein, coumarins, rhodamines, 5-TMRIA (tetramethylrhodamine-5-iodoacetamide), (9-(2(or 4)-(N-(2-maleimdylethyl)-sulfonamidyl)-4(or 2)-sulfophenyl)-2,3,6,7,12,13,16,17-octahydro-(1H,5H,11H,15H-xantheno(2,3,4-ij:5,6,7-i′j′)diquinolizin-18-ium salt) (Texas Red®), 2-(5-(1-(6-(N-(2-maleimdylethyl)-amino)-6-oxohexyl)-1,3-dihydro-3,3-dimethyl-5-sulfo-2H-indol-2-ylidene)-1,3-propyldienyl)-1-ethyl-3,3-dimethyl-5-sulfo-3H-indolium salt (Cy™3), N,N′-dimethyl-N-(iodoacetyl)-N′-(7-nitrobenz-2-oxa-1,3-diazol-4-yl)ethylenediamine (IANBD amide), 6-acryloyl-2-dimethylaminonaphthalene (acrylodan), pyrene, 6-amino-2,3-dihydro-2-(2-((iodoacetyl)amino)ethyl)-1,3-dioxo-1H-benz(de)isoquinoline-5,8-disulfonic acid salt (lucifer yellow), 2-(5-(1-(6-(N-(2-maleimdylethyl)-amino)-6-oxohexyl)-1,3-dihydro-3,3-dimethyl-5-sulfo-2H-indol-2-ylidene)-1,3-pentadienyl)-1-ethyl-3,3-dimethyl-5-sulfo-3H-indolium salt (Cy™5), 4-(5-(4-dimethylaminophenyl)oxazol-2-yl)phenyl-N-(2-bromoacetamidoethyl)sulfonamide (Dapoxyl® (2-bromoacetamidoethyl)sulfonamide)), (N-(4,4-difluoro-1,3,5,7-tetramethyl-4-bora-3a,4a-diaza-s-indacene-2-yl)iodoacetamide (BODIPY® 507/545 IA), N-(4,4-difluoro-5,7-diphenyl-4-bora-3a,4a-diaza-s-indacene-3-propionyl)-N′-iodoacetylethylenediamine (BODIPY 530/550 IA), 5-((((2-iodoacetyl)amino)ethyl) amino)naphthalene-1-sulfonic acid (1,5-IAEDANS), and carboxy-X-rhodamine, ⅚-iodoacetamide (XRIA 5,6). Another example of a label is BODIPY-FL-hydrazide. Other luminescent labels include lanthanides such as europium (Eu3+) and terbium (Tb3+), as well as metal-ligand complexes of ruthenium [Ru(II)], rhenium [Re(I)], or osmium [Os(II)], typically in complexes with diimine ligands such as phenanthroline.

Once levels of the known biomarker are measured, these measured levels are compared to normal levels of the biomarker to determine a difference, if any, between the measured levels and the normal levels of the biomarker. A difference between normal levels and the measured levels of the biomarker may indicate that the subject has a disease or abnormal condition or has a higher (or lower) probability of developing a disease or abnormal condition than normal subjects. In addition the magnitude of difference between measured levels and normal levels of the biomarker may also indicate the severity of disease or abnormal condition or the level of probability of developing a disease or abnormal condition, compared to normal subjects.

The difference between measured levels of the biomarker and normal levels may be a relative or absolute quantity. In addition, “levels of biomarkers” is used to mean any measure of the quantity of the biomarker such as, but not limited to, mass, concentration, biological activity. Example of biological activities that may be used to quantify biomarkers include, but are not limited to, chemotactic, cytotoxic, enzymatic or other biological activities, such as quantifiable activities that are used, for example, by the National Institute for Biological Standards and Control (NIBSC) in the United Kingdom for the quantification of interferon, cytokine and growth-factor activity. The difference in levels of biomarker may be equal to zero, indicating that the patient is normal, or that there has been no change in levels of biomarker since the previous assay. The difference may simply be, for example, a measured fluorescent value, radiometric value, densitometric value, mass value etc., without any additional measurements or manipulations. Alternatively, the difference may be expressed as a percentage or ratio of the measured value of the antigen to a measured value of another compound including, but not limited to, a standard, such as the SAP standard. The difference may be negative, indicating a decrease in the amount of measured biomarker over normal value or from a previous measurement, and the difference may be positive, indicating an increase in the amount of measured antigen over normal values or from a previous measurement. The difference may also be expressed as a difference or ratio of the biomarker to itself, measured at a different point in time. The difference may also be determined using in an algorithm, wherein the raw data is manipulated.

“Normal levels” of a given biomarker may be assessed by measuring levels of the biomarker in a known healthy subject, including the same subject that is later screened or being diagnosed. Normal levels may also be assessed over a population sample, where a population sample is intended to mean either multiple samples from a single patient or at least one sample from a multiple of subjects. Normal levels of a biomarker, in terms of a population of samples, may or may not be categorized according to characteristics of the population including, but not limited to, sex, age, weight, ethnicity, geographic location, fasting state, state of pregnancy or post-pregnancy, menstrual cycle, general health of the patient, alcohol or drug consumption, caffeine or nicotine intake and circadian rhythms.

The present invention also relates to methods of diagnosing or testing for an abnormal condition in a patient. As used herein the term “diagnose” means to confirm the results of other tests or to simply confirm suspicions that the patient may have a particular abnormal condition. A “test,” on the other hand, is used to indicate a screening method where the patient or the healthcare provider has no indication that the patient may, in fact, have a particular disease or particular abnormal condition. The methods of testing herein may be used for a definitive diagnosis, or the tests may be used to assess a patient's likelihood or probability of developing a disease or abnormal condition.

The methods of the present invention, therefore, may be used for diagnostic or screening purposes. Both diagnostic and testing can be used to “stage” a condition or disease in a patient. As used herein, the term “stage” is used to indicate that the abnormal condition or disease can be categorized, either arbitrarily or rationally, into distinct degrees of severity. The categorization may be based upon any quantitative characteristic that can be separated, such as, but not limited to, a numerical value of a biomarker, or it may be based upon qualitative characteristics that can be separated. The term “stage” may or may not involve disease progression. In addition, the assay or measurement may be used to stratify a population into relevant cohorts of similarly classified individuals, such as for a clinical trial or other study.

The present invention also relates to methods of monitoring the progression of an abnormal condition in a subject, as well as methods of monitoring the efficacy of a treatment or a potential treatment in a subject with an abnormal condition, with the methods comprising establish one or more standard curves, where the standard comprises a SAP peptide. The methods further comprise contacting more than one sample from a subject with at least one binding agent that is capable of binding an aggregated biomarker, where the multiple samples are taken from the subject at different time points. The level binding activity of the binding agent in the samples is detected and the binding activity in each sample is correlated to the established standard curve(s) to determine the levels of the aggregated biomarker in the subject. The determined levels of the aggregated biomarker from each time point, using SAP as a standard, are then compared to each other to determine if the measured levels of the aggregated biomarker are changing over time.

Thus, the present invention also relates to methods of screening potential therapeutics for their ability to prevent or reverse protein aggregation in vitro. The methods may comprise, for example, monitoring the rate of aggregation of a biomarker in the presence or absence of a test compound and determining if the test compound alters the rate of aggregation. The SAPs of the present invention, could, of course, be used to establish binding curves and aggregation rate curves as well.

The invention may also be used to screen antibodies that have been developed as potential therapeutics, such as, but not limited to, humanized antibodies. Currently, vaccination studies are underway that have the intent of generating antibodies in the subject that bind and antagonize the effect of aggregated beta amyloid, and possibly promote the clearance of beta amyloid aggregates. The administration of humanized antibodies raised against aggregates of beta amyloid, as well as other proteins or peptides that have a tendency to form toxic aggregates, may permit active immunization programs to be circumvented. The compositions of the present invention may be used to compare the affinity or other characteristics of generated antibodies.

Similarly, the SAPs of the present invention may also be used as vaccinations themselves. Accordingly, the SAPs, which may less toxic or even non-toxic to the host cell or organism, may be administered in such a manner as to elicit an immune response from the cell or organism, while reducing risks associated with administering traditional vaccines. The vaccines may be in the form of single dose preparations or in multi-dose flasks which can be used for mass vaccination programs. Reference is made to Remington's Pharmaceutical Sciences, Mack Publishing Co., Easton, Pa., Osol (ed.) (1980); and New Trends and Developments in Vaccines, Voller et al. (eds.), University Park Press, Baltimore, Md. (1978), for methods of preparing and using vaccines. Thus, in one embodiment, the present invention provides for methods of vaccinating a subject, with the method comprising administering to the subject a protection-inducing amount of a SAP vaccine, with the vaccine comprising a SAP and an adjuvant. In specific embodiments, the SAP of the SAP vaccine is a MAP. In even more specific embodiments, the MAP of the SAP vaccine is comprises at least a portion of the Aβ peptide, the huntingtin peptide, the alpha-synuclein peptide, the SOD-1 peptide, islet amyloid polypeptide and the prion peptide or mutants thereof.

The following examples are for illustrative purposes and are not intended to limit the scope of the subject matter of the present invention.

### EXAMPLES

### Example 1

### Preparation of a 4-Branched MAP-Aβ₁₋₂₀

The MAP-Aβ₁₋₂₀ peptide was constructed using Fmoc protein synthesis chemistry, which is described in Tam, J. P. and Lu, Y.-A., Proc. Nat'l. Acad. Sci., 85:9084-9088 (1989); Ahlborg, N., J. Immunol. Methods, 179:269-275 (1995); and Espanel, X., et al., J. Biol. Chem. 278(17):15162-15167 (2003), all of which are incorporated by reference in their entirety. The β-alanine was first immobilized, and the lysine residues were added to the immobilized β-alanine Through a series of addition of protected amino acid residues, the chains were elongated in the C to N terminal direction.

### Example 2

### Quantification of Aggregated Aβ₁₋₄₂ Using MAP-Aβ₁₋₂₀

As described in U.S. Pat. Nos. 6,696,304, 6,649,414, 6,632,536 and 6,599,331, which are incorporated by reference, antibody specific for Aβ1-20 was conjugated to color encoded beads, composed of polystyrene, which were licensed from Luminex™ Corporation (Austin, Tex.). Wells of a 96-well plate were pre-wet with 200 μL working buffer/wash solution. The wash solution is available from Biosource International (Camarillo, Calif., USA). After about 15 to 30 seconds, the wash solution was aspirated from the wells using vacuum manifold.

The bead conjugation method used yields a 100× stock solution, containing approximately 20×10⁶ beads/mL. The beads used in the assay were prepared from the 100× stock solution. Just prior to use, the stock solution was vortexed for 30 seconds, and then sonicated for 30 seconds. The working solution of the conjugated beads, containing about 2×10⁵ beads/mL, was prepared by diluting the stock solution in wash buffer 1:100. Just prior to use, the working solution of conjugated beads was again vortexed for 30 seconds and sonicated for 30 seconds. About 25 μL (5000 beads) of conjugated bead solution was added to each well designated for the assay (including wells designated for the standard curve and for the samples) and the wells were subsequently shielded from light.

Next, 200 μL of wash solution was added to each well and the beads were allowed to soak for about 15 to 30 seconds. The wash solution was then aspirated with the vacuum manifold. The washing step was repeated. The residual liquid on the bottom of the plate was blotted on a clean paper towel.

The MAP-Aβ₁₋₂₀ standards were prepared in the following concentrations: 20 ng/mL; 6.67 ng/mL, 2.22 ng/mL, 0.74 ng/mL, 0.25 ng/mL, 0.082 ng/mL; 0.027 ng/mL and a blank. Each well designated for standard received 100 μL of standard.

Next, 50 μL buffer was pipetted into each of the well and then 50 μL of each sample was pipetted into designated wells in duplicate. The plate was incubated for about 2 hours at room temperature on an orbital shaker at about 500-600 rpm. After incubation, the liquid was aspirated from the wells with a vacuum manifold at a pressure of less than about 5 inches Hg. The wells were washed three times with 200 μl of wash solution buffer.

For detection, 100 μL of a biotinylated detector antibody at a concentration of about 5 μg/mL, was added to each well the plate and incubated for about 1 hour at room temperature on an orbital shaker at about 500-600 rpm. The detector antibody is specific for an epitope of the Aβ₁₋₂₀.

Ten to fifteen minutes before the end of the incubation period, a streptavidin-R-Phycoerythrin solution was prepared. The concentration of the steptavidin-R-Phycoerythrin was about 12 μg/mL.

After the 1 hour incubation, the biotinylated detector antibody solution was aspirated from the wells with a vacuum manifold at a pressure of less than about 5 inches Hg. The beads were washed three times and the residual liquid was blotted from the bottom of the plate on clean paper towels.

After removal of the detector antibody solution and subsequent washing, about 100 μL of the streptavidin-R-Phycoerythrin solution was added to each well and incubated for about 30 minutes at room temperature on an orbital shaker at 500-600 rpm.

The streptavidin-R-Phycoerythrin solution was aspirated from the wells using a vacuum manifold at a pressure of less than about 5 inches Hg, and the wells were washed four times.

After washing, the beads were resuspended in buffer solution and fluorescence was read on a Luminex 100™.

From the known concentrations of MAP-Aβ₁₋₂₀ and the corresponding fluorescence values, a standard curve was generated using standard curve fitting software SOFTmaxPro. From the generated standard curve, concentration of samples were determined and then multiplied by 2 to correct for the 1:2 dilution in the wells.

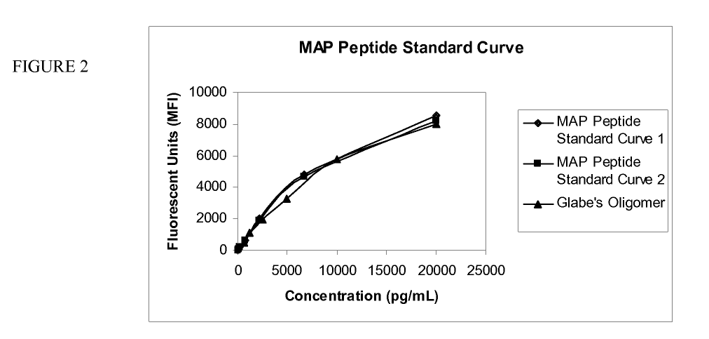

FIG. 2 provides representative standard curves obtained in the aggregated Aβ assay for the Luminex™ platform, using the MAP-Aβ₁₋₂₀ as the standard.

Also depicted in FIG. 2 is the comparison of the reactivity of the MAP-Aβ₁₋₂₀ with the reactivity of the indicated concentrations of Glabe's oligomer. In this example, Glabe's oligomer is an aggregated form of Aβ that is synthesized in vitro that is postulated to have a similar conformation to the natural Aβ aggregates found in biological samples. The following reference describes the production of Glabe's oligomer and is incorporated by reference: Demuro, A., J. Biol. Chem. 280(17):17294-17300 (2005).

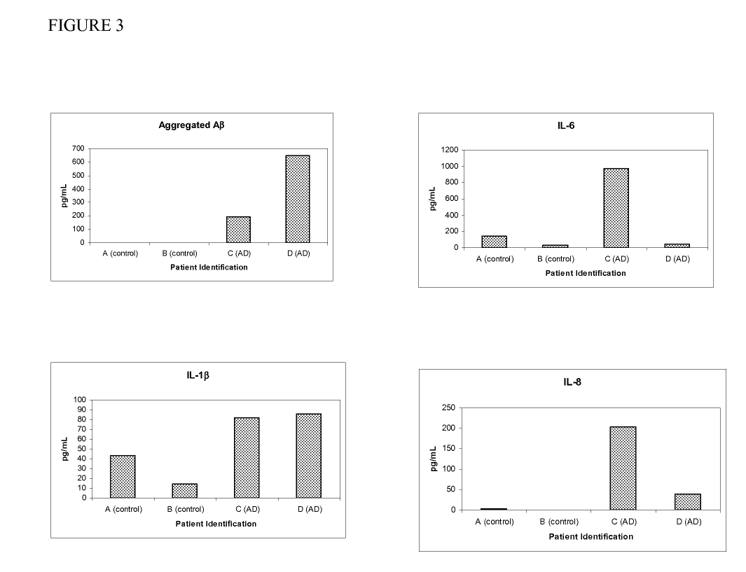

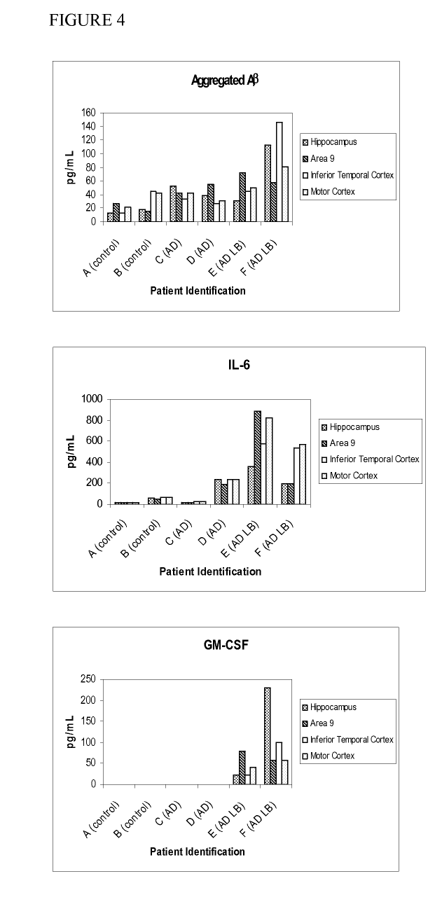

FIGS. 3 and 4 depict levels of natural aggregated Aβ in samples, using the MAP-Aβ₁₋₂₀ as a standard. FIG. 3 depicts the detection of natural aggregated Aβ in ventricular fluid samples from a cohort of elderly patients with Alzheimer's disease and elderly, non-demented control patients. The samples of FIG. 3 were collected into tubes, centrifuged briefly to sediment cells contained in the samples, then frozen until analyzed for aggregated Aβ with the aggregated Aβ assay described here.

Also depicted in FIG. 3 are correlations of concentrations of Aβ with concentrations of inflammatory cytokines that were measured with commercially available reagents from BioSource International, Inc. FIG. 4 depicts the detection of natural aggregated Aβ in tissue homogenates prepared from various brain regions of patients with Alzheimer's disease, patients with Alzheimer's disease with Lewy Bodies, and elderly, non-demented controls, using the assay described here. The samples of FIG. 4 were collected, weighed, homogenized in Tris buffered saline supplemented with protease inhibitors, then centrifuged for 1 hour at 100,000×g at 4° C. This procedure has been shown to minimize Aβ fibrils and protofibrils. The following references have used an ultracentrifugation step to eliminate Aβ fibrils from samples and are incorporated by reference: Gong, Y., et al., Proc. Natl. Acad. Sci. 199(18):10417-10422; Barghorn, S., et al. J. Neurochem. 95(3):834-847 (2005). Following the centrifugation step, the clear liquid that formed the middle layer of the sample was carefully extracted using a syringe to avoid the upper fatty layer and the pellet that comprised the bottom layer of the. Samples prepared in this manner were assayed with the aggregated Aβ assay described here, and concentrations of Aβ were correlated with concentrations of inflammatory cytokines, measured with commercially available reagents from BioSource, International, Inc.

### Example 3

### Preparation of a 4-Branched MAP-Alpha-Synuclein

The MAP-alpha-synuclein 116-130 peptide was constructed using Fmoc protein synthesis chemistry, which is described in Tam, J. P. and Lu, Y.-A., Proc. Nat'l. Acad. Sci., 85:9084-9088 (1989); Ahlborg, N., J. Immunol. Methods, 179:269-275 (1995); and Espanel, X., et al., J. Biol. Chem. 278(17):15162-15167 (2003), all of which are incorporated by reference in their entirety. As used herein, the phrase MAP-alpha-synuclein 116-130 peptide indicates a peptide with amino acids 116-130 of SEQ ID NO:2. Following the same general construction of the 4-branched MAP-Aβ₁₋₂₀ standard, a β-alanine moiety was first immobilized, and the lysine residues were added to the immobilized β-alanine. Through a series of addition of protected amino acid residues, the chains were elongated in the C to N terminal direction.

### Example 4

### Time Course Aggregation of Alpha-Synuclein

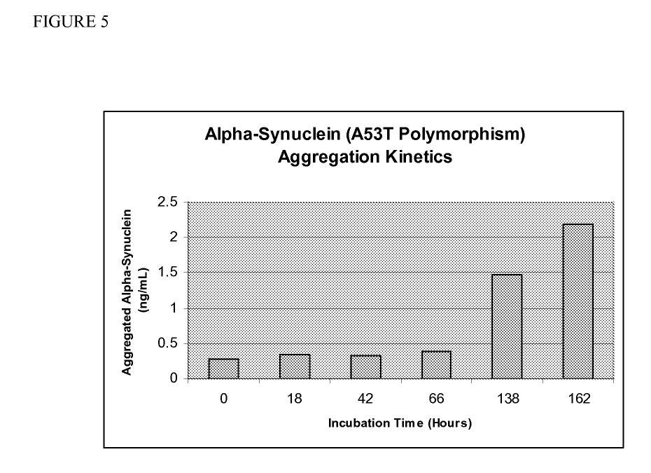

Aggregated Alpha-Synuclein was generated according to the following procedure. Recombinant Alpha-Synuclein A53T (Recombinant Peptide Technologies Cat. # S-1002-2) was reconstituted with deionized water to a concentration of 1 mg/mL. An aliquot (100 μL) of the recombinant protein was then dispensed into a 2 mL Corning Cryogenic vial and diluted to a final concentration of about 100 μg/mL in PBS containing 0.02% sodium azide. The vial was then capped, sealed with Parafilm, and placed on a rocker in a 37° C. incubator. At various times, aliquots of the protein were removed from the mixture, and stored at −20° C. At the completion of the incubation step, samples were defrosted at room temperature, then diluted to a final concentration of 1 μg/mL in Assay Diluent. The diluted samples were then assayed using the 211 mAb (Invitrogen Corp., Carlsbad, Calif., USA, Cat. #32-8100) as both the capturing and detecting (biotinylated) antibody. The results are presented in the FIG. 5.

### Example 5

### Quantification of Aggregated Alpha-Synuclein Using MAP-Alpha-Synuclein

As described in U.S. Pat. Nos. 6,696,304, 6,649,414, 6,632,536 and 6,599,331, which are incorporated by reference, antibody specific for alpha-synuclein was conjugated to color encoded beads, composed of polystyrene, which were licensed from Luminex™ Corporation (Austin, Tex.). Wells of a 96-well plate were pre-wet with 200 μL working buffer/wash solution. The wash solution is available from Biosource International (Camarillo, Calif., USA). After about 15 to 30 seconds, the wash solution was aspirated from the wells using vacuum manifold.

The bead conjugation method used yields a 100× stock solution, containing approximately 20×10⁶ beads/mL. The beads used in the assay were prepared from the 100× stock solution. Just prior to use, the stock solution was vortexed for 30 seconds, and then sonicated for 30 seconds. The working solution of the conjugated beads, containing about 2×10⁵ beads/mL, was prepared by diluting the stock solution in wash buffer 1:100. Just prior to use, the working solution of conjugated beads was again vortexed for 30 seconds and sonicated for 30 seconds. About 25 μL (5000 beads) of conjugated bead solution was added to each well designated for the assay (including wells designated for the standard curve and for the samples) and the wells were subsequently shielded from light.

Next, 200 μL of wash solution was added to each well and the beads were allowed to soak for about 15 to 30 seconds. The wash solution was then aspirated with the vacuum manifold. The washing step was repeated. The residual liquid on the bottom of the plate was blotted on a clean paper towel.

The MAP-alpha synuclein standards were prepared in the following concentrations: 0.738 ng/mL; 0.246 ng/mL, 0.0819 ng/mL and 0.0273 ng/mL, in addition to a blank. Each well designated for standard received 100 μL of standard.

Next, 50 μL buffer was pipetted into each of the well and then 50 μL of each sample was pipetted into designated wells in duplicate. The plate was incubated for about 2 hours at room temperature on an orbital shaker at about 500-600 rpm. After incubation, the liquid was aspirated from the wells with a vacuum manifold at a pressure of less than about 5 inches Hg. The wells were washed three times with 200 μL of wash solution buffer.

For detection, 100 μL of a biotinylated detector antibody at a concentration of about 2 μg/mL, was added to each well the plate and incubated for about 1 hour at room temperature on an orbital shaker at about 500-600 rpm. The detector antibody (211 mAb) is specific for an epitope of alpha synuclein.

Ten to fifteen minutes before the end of the incubation period, a streptavidin-R-Phycoerythrin solution was prepared. The concentration of the steptavidinR-Phycoerythrin was about 5 μg/mL.

After the 1 hour incubation, the biotinylated detector antibody solution was aspirated from the wells with a vacuum manifold at a pressure of less than about 5 inches Hg. The beads were washed three times and the residual liquid was blotted from the bottom of the plate on clean paper towels.

After removal of the detector antibody solution and subsequent washing, about 100 μL of the streptavidin-R-Phycoerythrin solution was added to each well and incubated for about 30 minutes at room temperature on an orbital shaker at 500-600 rpm.

The streptavidin-R-Phycoerythrin solution was aspirated from the wells using a vacuum manifold at a pressure of less than about 5 inches Hg, and the wells were washed four times.

After washing, the beads were resuspended in buffer solution and fluorescence was read on a Luminex 100™.

From the known concentrations of MAP-alpha-synuclein and the corresponding fluorescence values, a standard curve was generated using standard curve fitting software SOFTmaxPro. From the generated standard curve, concentration of samples were determined and then multiplied by 2 to correct for the 1:2 dilution in the wells.

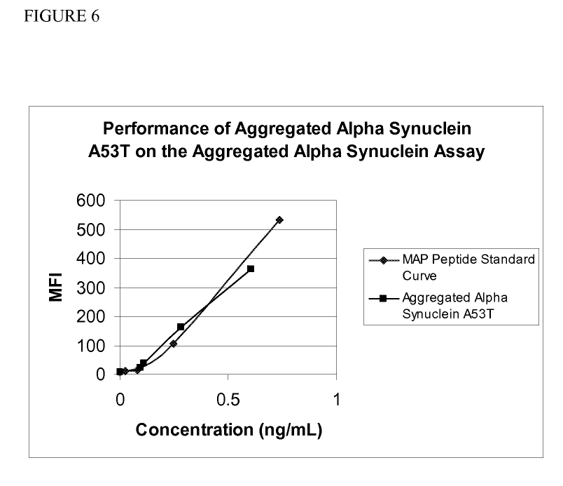

FIG. 6 provides representative standard curves obtained in the aggregated alpha-synuclein assay for the Luminex™ platform, using the MAP-alpha-synuclein as the standard.

Also depicted in FIG. 6 is the comparison of the reactivity of the MAP-alpha-synuclein with the reactivity of the indicated concentrations of laboratory-aggregated alpha-synuclein from Example 4.

What is claimed is:

1. A method for quantifying a known biomarker in a sample, said method comprising an assay comparing the binding activity of a binding agent to said known biomarker with the binding activity of said binding agent to a synthetic aggregated peptide (SAP).

2. The method of claim 1 wherein said known biomarker is a peptide.

3. The method of claim 2, wherein said known peptide is selected from the group consisting of beta amyloid (Aβ), huntingtin, alpha-synuclein, superoxide dismutase-1, (SOD1) and prion peptide.

4. The method of claim 3, wherein said known peptide is an aggregated oligomer.

5. The method of claim 4, wherein said aggregated oligomer is an aggregated oligomer of Aβ.

6. The method of claim 3, wherein said binding agent is an antibody or functional fragment thereof.

7. The method of claim 6, wherein said assay is an assay selected from the group consisting of a colorimetric assay and a radiometric assay.

8. The method of claim 7, wherein said assay is a colorimetric assay that is an enzyme-linked immunosorbence assay (ELISA).

9. The method of claim 8, wherein said SAP is a multiple antigenic peptide (MAP).

10. The method of claim 9, wherein said MAP with more than 4 branches.

11. The method of claim 9, wherein said MAP is a 4-branched MAP.

12. The method of claim 11, wherein said MAP comprises at least a portion of a peptide selected from the group consisting of beta amyloid (Aβ), huntingtin, alpha-synuclein, superoxide dismutase-1, (SOD1) and prion peptide.

13. The method of claim 11, wherein said MAP comprises at least a portion of the Aβ peptide.

14. The method of claim 13, wherein said portion of Aβ is the N-terminus said Aβ peptide.

15. The method of claim 14, wherein said N-terminus of said Aβ comprises amino acids 1-10 of SEQ ID NO. 1.

16. The method of claim 15, wherein said N-terminus of said Aβ comprises amino acids 1-20 of SEQ ID NO:1.

17. The method of claim 11, wherein said MAP comprises at least a portion of the alpha-synuclein peptide.

18. The method of claim 17, wherein said portion of alpha-synuclein is near the C-terminus said alpha synuclein peptide.

19. The method of claim 18, wherein said C-terminus of said alpha-synuclein comprises amino acids 121-125 of SEQ ID NO. 2.

20. The method of claim 19, wherein said N-terminus of said alpha-synuclein comprises amino acids 116-130 of SEQ ID NO. 2.

21. A method of detecting an abnormal condition in a subject, said method comprising
a) detecting the binding activity of a binding agent towards at least one standard to establish a standard curve, said standard comprising a synthetic aggregated peptide (SAP);
b) contacting a sample from said subject with at least one binding agent that is capable of binding a biomarker, wherein said biomarker is an aggregated biomarker;
c) detecting the level binding activity of said binding agent in said sample;
d) correlating said level of binding activity in said sample to said standard curve to determine the levels of said aggregated biomarker in said subject; and
e) comparing the levels of said aggregated biomarker in said subject to normal levels of said aggregated biomarker to determine a difference between measured levels of said aggregated biomarker and normal levels of said aggregated biomarker;
wherein a difference between said measured levels of said aggregated biomarker and said normal levels of said aggregated biomarker, is indicative of an abnormal condition in said subject.

22. The method of claim 21, wherein said abnormal condition is selected from the group consisting of Alzheimer's Disease, Huntington's Disease, Parkinson's Disease, Cruetzfeldt-Jakob Disease, and heart disease or any stage thereof.

23. The method of claim 22, wherein said aggregated biomarker is selected from the group consisting of aggregated beta amyloid (Aβ), aggregated huntingtin, aggregated alpha-synuclein, aggregated superoxide dismutase-1, (SOD1) and aggregated prion peptide.

24. The method of claim 23, wherein said SAP is a multiple antigenic peptide (MAP).

25. The method of claim 24 wherein said MAP comprises 4 branches.

26. The method of claim 25 wherein at least one branch of said MAP standard comprises at least a portion of a peptide selected from the group consisting of beta amyloid (Aβ), huntingtin, alpha-synuclein, superoxide dismutase-1, (SOD1) and prion peptide.

27. The method of claim 26 wherein said abnormal condition is Alzheimer's Disease, said aggregated biomarker is aggregated Aβ and at least one branch of said MAP standard comprises at least a portion of the beta amyloid (Aβ) peptide.

28. The method of claim 27 wherein said at least one branch of said MAP standard comprises the N-terminus of said amyloid beta (Aβ) peptide.

29. The method of claim 28 wherein said N-terminus of Aβ peptide comprises amino acids 1-20 of SEQ ID NO:1.

30. The method of claim 26 wherein said abnormal condition is Parkinson's Disease, said aggregated biomarker is aggregated alpha-synuclein and at least one branch of said MAP standard comprises at least a portion of an alpha-synuclein peptide.

31. The method of claim 30 wherein said at least one branch of said MAP standard comprises a portion of the C-terminus of said alpha-synuclein peptide.

32. The method of claim 31, wherein said C-terminus of said alpha-synuclein peptide comprises amino acids 121-125 of SEQ ID NO:2.

33. A composition comprising a branched MAP peptide, wherein at least one branch of said MAP peptide comprises the N-terminus of amyloid beta (Aβ) peptide.

34. The composition of claim 30, wherein said MAP peptide comprises 4 branches.

35. The composition of claim 31, wherein each of said 4 branches comprises said N-terminus of Aβ peptide.

36. The composition of claim 32, wherein said N-terminus of Aβ peptide comprises amino acids 1-20 of SEQ ID NO:1.

37. The peptide of claim 33, wherein at least one of said 4 branches comprises a peptide other than said N-terminus of Aβ peptide.

38. A composition comprising a branched MAP peptide, wherein at least one branch of said MAP peptide comprises a portion of the C-terminus of alpha-synuclein peptide.

39. The composition of claim 38, wherein said MAP peptide comprises 4 branches.

40. The composition of claim 39, wherein each of said 4 branches comprises said portion of said C-terminus of alpha-synuclein peptide.

41. The composition of claim 30, wherein said portion of said C-terminus of alpha-synuclein peptide comprises amino acids 121-125 of SEQ ID NO:1.

42. The peptide of claim 41, wherein at least one of said 4 branches comprises a peptide other than said portion of said C-terminus of alpha-synuclein peptide.
